(19) United States
(12) Patent Application Publication
(10) Pub. No.: US 2006/0159773 A1
(43) Pub. Date: Jul. 20, 2006

(54) Herbal compositions containing hoodia

(76) Inventor: Stephen Holt, Little Falls, NJ (US)

Correspondence Address: LUCAS & MERCANTI, LLP, 475 PARK AVENUE SOUTH, 15TH FLOOR, NEW YORK, NY, 10016, US

(21) Appl. No.: 11/336,155

(22) Filed: Jan. 20, 2006

Related U.S. Application Data
(60) Provisional application No. 60/645,445, filed on Jan. 20, 2005.

(51) Int. Cl. A61K 36/185; A61K 31/555; A61K 36/82; A61K 33/26

(52) U.S. Cl. 424/646; 424/769; 424/729; 424/776; 514/184; 514/456

(57) ABSTRACT

The present invention relates to herbal compositions containing the Hoodia gordonii and synergistically enhancing ingredients such as green coffee bean extract. The compositions are useful in controlling obesity and supporting the treatment of various health conditions related thereto, including Syndrome X. Methods of treatment using the compositions described herein are also disclosed.

### CROSS-REFERENCE TO RELATED APPLICATIONS

This application claims the benefit of priority from U.S. Provisional Application Ser. No. 60/645,445, filed Jan. 20, 2005, the contents of which are incorporated herein by reference.

### FIELD OF THE INVENTION

The present invention relates to herbal compositions containing the Hoodia gordonii cactus (genus Trichocaulon) and synergistically acting ingredients. The invention also relates to methods of controlling obesity, appetite, and supporting the treatment of metabolic conditions including obesity, Syndrome X, high blood pressure and the like.

### BACKGROUND OF THE INVENTION

Hoodia gordonii is a “stinky” plant from South Africa with miraculous properties. This botanical contains substances that may exert dramatic control over hunger, appetite, and thirst. Although the Hoodia gordonii plant looks like a cactus, it belongs to a category of plants called succulents. The use of Hoodia as an appetite suppressant is supported by colorful folklore history and recent scientific studies. Hoodia is well known to the San bushmen of South Africa, who learned to eat this plant from their forefathers. The San bushmen believe that Hoodia is their food, water, and medicine.

The Hoodia plant grows in the Kalahari desert region of South Africa, including Namibia, Angola, and Botswana. The San people of the Kalahari desert used the Hoodia plant to reduce hunger and thirst when they traveled across the desert. This nomadic, ancient group of people was often deprived of food on long hunting expeditions in the desert. In the 1937, a Dutch anthropologist discovered the traditional use of the Hoodia plant by the San bushmen. More recently, interest has turned to identifying specific portions of the plant such as extracts and/or sterol glycosides as the active portion of the plant responsible for appetite suppression. See, for example CSIR's U.S. Pat. No. 6,376,657, the contents of which are incorporated herein by reference.

While the identification of specific compounds found in the plant have been an advance in the understanding of how the Hoodia plant controls hunger, early clinical results relating to the administration of specific sterol glycosides have not met expectations. This has therefore led some to consider whether the desirable effects provided by Hoodia are attributable to a combination of ingredients found in the plant.

United States Patent Application Publication No. 2004/0265398 (the contents of which are incorporated herein by reference) describes the use of the Hoodia plant itself, preferably in the form of sun-dried chips or 80 mesh powder, alone or combined with a blend of beneficial herbs and compounds, rather than a chemical extract thereof for the purposes of controlling obesity. One of the keys to the treatment described therein is said to be in the timing and duration of the Hoodia administration. While this return to the use of whole Hoodia rather than extracts thereof begins to address one of the shortcomings associated with administering a single compound isolated from the plant, further research is required. Specifically, there has been a need in the art to provide Hoodia-based compositions and methods of treatment which provide enhanced treatment benefits for certain metabolic conditions. The present invention addresses this need.

### SUMMARY OF THE INVENTION

In one aspect of the invention there is provided novel herbal-based compositions comprising
a) a weight reducing amount of Hoodia gordonii or an extract thereof, and
b) an effective amount of green coffee bean extract.

A further aspect of the invention includes methods of inducing weight loss and/or controlling appetite in mammals by administering an effective amount of a composition described herein to a mammal in need of such treatment.

In another aspect of the invention, there is provided a composition comprising a) a weight reducing amount of Hoodia gordonii or an extract thereof; and b) an effective amount of chlorogenic acid. Preferably, the extract of Hoodia gordonii is a sterol glycoside such as P57.

Still further aspects of the invention include methods of treating Type II diabetes mellitus, treating metabolic Syndrome X and related metabolic problems found in metabolic syndromes and methods of increasing the effectiveness of an anti-diabetic compound in mammals. In each case, the methods include administering an effective amount of a Hoodia-containing composition as described herein alone or in combination with the varying combinations of pharmacologic/chemical and/or herbal agents described herein or an anti-diabetic compound to a mammal in need of such treatment.

Other and further aspects of the invention will be apparent from the detailed description and claims following below.

A few of the unexpected advantages provided by the present invention are described below.

The combination of Hoodia gordonii with GCBE provides unique and global advantages. The principle component of GCBE that is a focus of the present invention is the content of chlorogenic acid in coffee beans or extracts. There are other polyphenol molecules in coffee beans which may provide added advantages in the combination products described herein. The recognition that obesity is commonly associated with the metabolic Syndrome X supports the added value of the combinations of Hoodia gordonii and GCBE. Hoodia gordonii has been proposed in the literature as a potentially valuable approach to the management of the metabolic Syndrome X. The main mechanisms of action of Hoodia gordonii in the metabolic Syndrome X is appetite suppression, leading to control of calorie intake. The ensuing weight loss would at least partially improve the constellation of problems found in the metabolic Syndrome X and its associated diseases. Improvements in weight control would result in potential improvements in the promotion of healthy blood cholesterol and a combat, at least in part, against insulin resistance. Weight control would also promote a lowering of blood pressure in the presence of hypertension. By adding unique components found in GCBE, including but not limited to chlorogenic acid, and other phytochemicals found in GCB, one would further impact the constellation of problems in the metabolic Syndrome X or pre-diabetic state or obesity-related pathophysiology. Chlorogenic acid has a potent and versatile effect on glucose metabolism, with an impact on the function of insulin, together with an impact on fat storage by the body, and potentially favorable outcomes in individuals with Type II diabetes mellitus. Chlorogenic acid inhibits glucose metabolism and absorption. Scientific studies show that chlorogenic acid inhibits portions of the glucose-6-phosphatase enzyme systems with a specific effect on glucose-6-phosphatase translocase. Chlorogenic acid may reduce glucose release from the liver which is often abnormally high in individuals with obesity, the metabolic Syndrome X, and Type II diabetes mellitus. Therefore, the combination of Hoodia gordonii with GCBE adds new and novel advantages for the management of obesity, obesity-related disorders, and metabolic problems associated with obesity. GCBE can be used without caffeine or with caffeine. The value of GCBE is underscored by several clinical observations that coffee consumption is associated with a lower occurrence of Type II diabetes mellitus. A significant advantage of the proposed combination supplement is to attack the clear, documented linkage between obesity and the prevalence of Type II diabetes mellitus. In this regard, the unique combination of Hoodia gordonii and GCBE has value in the prevention of Type II diabetes mellitus, as well as the management of obesity, glucose intolerance, and associated metabolic disorders and diseases. Cinnamon and cinnamon extracts having insulin-mimetic properties can, as will be described in detail below, also be included with the Hoodia-based compositions described herein.

It has been surprisingly found that combinations of Hoodia gordonii and CGBE-containing chlorogenic acid or Hoodia gordonii and chlorogenic acid per se reduce insulin levels in Type II diabetes mellitus patients, patients with pre-diabetes, the metabolic Syndrome X and related conditions. On the basis of open label observations, the reductions are more than that observed when either is given alone and, in fact, such combinations provide a synergistic action to provide benefits beyond the results expected.

### DETAILED DESCRIPTION OF THE INVENTION

In preferred aspects of the invention, the Hoodia included in the compositions and methods described herein is obtained from the whole plant. It is preferably processed by drying the plant, and thereafter producing powder or concentrates therefrom using standard processing techniques known to those of ordinary skill and without requiring undue experimentation. It is particularly preferred if chemical extraction techniques are avoided.

The rational use of Hoodia in a dietary supplement involves the taking of a powder, capsule, or tablet of material produced from the whole plant. There seems to be no doubt that constituents in Hoodia with biological activity are regularly absorbed from the digestive tract. In other words, the oral ingestion of Hoodia plant would be expected to have effects on the body similar to those described by the San bushmen, as long as the components of Hoodia that are responsible for its effects are not destroyed during processing. There are many possible ways of processing the Hoodia plant. Some of these methods of processing have been disclosed in patents, and some involve the arbitrary use of drying techniques, sterilization techniques, and in some cases, the use of chemicals to concentrate components of Hoodia. One preferred source of the Hoodia included in the compositions of the invention is supplied by Stella Labs, Inc. of New Jersey, through growers of Hoodia in South Africa such as those associated with Synhealth Corporation of South Africa or other such similar corporations or recognized suppliers of Hoodia located in South Africa and other countries known to be capable of supplying the same. Regardless of the source, the Hoodia gordonii is preferably in powdered form derived from the whole Hoodia gordonii plant for the preferred compositions of the invention.

The principal ingredient included with the Hoodia is a green coffee bean extract (GCBE) which contains chlorogenic acid. One such product contemplated for use herein is available from Optipure, Chemco Industries, Inc. and is a substantially non-caffeine containing composition obtained by aqueous ethanol extraction of green coffee beans. Some of the main compounds found in the extract include quinic acid, caffeic acid, p-coumaric acid and, of course, chlorogenic acid. Other sources of chlorogenic acid are also contemplated and will be present in the form of standardized extracts containing from about 10-50% or greater chlorogenic acid by weight. Organically synthesized chlorogenic acid can be used in addition to or in place of the GCBE-based extract.

While not wishing to be bound by theory, Applicant submits that the synergistic benefits of combining the Hoodia and green coffee bean extract is due, at least in part, to the fact the therapeutic mechanisms of action for each ingredient is separate and apart from the other. The Hoodia is believed to act in large part by giving the hypothalamus a powerful satiety signal. This signal confers a feeling of fullness to the person ingesting the herb. Increased nerve cell activity in the hypothalamus was also observed after administration of Hoodia. The green coffee bean extract is believed to principally act by inhibiting the absorption of both fat and sugar and enhancing fat metabolism. Since it is substantially free of caffeine, the GCBE exerts its weight loss effects without causing a stimulant effect on the patient. This is to be contrasted with other herbal enhancers which were proposed for combination with Hoodia which sought to supplement the activity of the Hoodia by attempting to increase metabolism, heart rate, pulse, etc. Such peripheral effects can have negative health implications in some patients, especially those who are obese with high blood pressure and/or others with Syndrome X, cardiovascular disease, insulin resistance, etc.

In book Supreme Properties of Hoodia Gordonii by Stephen Holt, the herb is postulated to have effects on the central nervous system that could alter appetite or sugar metabolism by actions of peripheral tissues of the body or organs such as the liver. Specifically, the glycosides in Hoodia favorably affect sugar metabolism or storage in the liver.

The unique combination of Hoodia and GCBE takes advantage of complementary but largely separate mechanisms of action in the body. These dual modes of activity are joined to provide a single composition that has unexpectedly improved desirable results. Because the effect of the Hoodia does not completely shut off the person's desire to eat, for example due to psychological reasons, it has been found that more beneficial therapeutic results can be had when the Hoodia is combined with a composition which combats obesity in a completely different manner. The ability of the GCBE to delay fat absorption or accumulation and increase lipolysis activity provides the patient with an effective supplement to combat obesity in combination with Hoodia's powerful satiety signal to the hypothalamus.

In view of the foregoing, there is provided in one aspect of the invention compositions comprising:
a) a weight reducing amount of Hoodia gordonii or an extract thereof; and
b) an effective amount of green coffee bean extract.

For purposes of the present invention, the term “weight reducing amount” shall be understood to mean an amount which is effective to achieve, even temporarily, the satiety feeling in persons or animals taking the same. It will be apparent to those of ordinary skill that the amounts required for achieving this effect will vary depending upon the size, weight, age, etc. of the species of the recipient. The amount is also dependent upon the ultimate dosage form and length of treatment time desired.

For purposes of the invention, the term “effective amount” shall be understood to mean an amount or quantity of the GCBE that is required to cause the metabolic effects described herein, i.e. delay fat absorption, increase lipolysis activity. This amount is readily determined by observation both before and after administration of the compositions described herein.

For purposes of the present invention, “weight-reducing” and “effective” amounts are synonymous with a “pharmaceutically or clinically effective amounts”. Such amounts, as known to those of ordinary skill are the doses or amounts that effective in treating or ameliorating symptoms and/or signs of obesity, Syndrome X, etc. in the subject being given the compositions of the invention. These later amounts are also readily determined by one of ordinary skill in the art, e.g., by observing or detecting changes in clinical condition of their patients. See in particular however, Combat Syndrome X, Y, and Z . . . , Stephen Holt, M.D., www.WellnessPublishing.com, the contents of which are included herein by reference.

In one preferred embodiment of the invention, the Hoodia-based combinations described herein are included in methods of treating metabolic syndromes, including Syndrome X, pre-diabetes, diabetes prevention, established diabetes mellitus, as well as diseases associated with these disorders that can be treated or improved by weight reduction and/or alteration of fat or sugar metabolism in mammals. Such associated conditions include but are not limited to polycystic ovary syndrome and its variants or associated conditions in the presence or absence of diabetes mellitus. Other diseases associated with the combination of metabolic problems found in the metabolic syndrome or associated with obesity may be improved or prevented by the varying combinations of chemical agents or biologically effective compositions described herein and the above-mentioned Combat Syndrome X, Y and Z, including specifically cardiovascular disease related to atherosclerosis or atheroma, Alzheimer's disease, peripheral neuropathy, and cognitive decline due to brain or CNS disorders with the specific secondary reduction of inflammation associated with the metabolic syndrome.

In accordance with the foregoing, the compositions of the present invention are preferably administered in combination with complementary biologically active herbal, homeopathic or pharmacologic agents. One particularly useful combination therapy includes Syndrome X Nutritional Factors® (available from Natures Benefit, Little Falls, N.J.) or other combinations of herbal compositions including, but not limited to:

Soluble fiber, soy protein, chromium, alpha lipoic acid, coffee bean extract, fish oils, vanadium, starch blockers, cinnamon, maitake, antioxidants, green tea extracts, etc. and the like.

Herbs and botanicals such as those mentioned in Combat Syndrome X, Y and Z, on pp 269-270, having beneficial effects on glucose intolerance and optionally, cholesterol control. A non-limiting list of suitable botanicals include Tinospora cordifolia, Pterocarpus marsupium, Azadirachta indica, Ficus racemosa, Aegle marmelose (possibly), Syzygium cumini, Cinnamonum tamala, Atriplex halimu, Vaccinium myrtillus, Korean ginseng, Opuntia ficus, Ocimum sanctum, Silybum marianim, etc.

The inventive Hoodia-based compositions can also include cinnamon or cinnamon extracts. Cinnamon is known to contain compounds that have effects on altering blood sugar metabolism including compounds that are insulin mimetic in a whole form or extracted form to contain enriched fractions of chalcones, and/or polyphenols, including one particular extract of cinnamon, methyl hydroxy chalcone polymer (MHCP). Other hydroxyl- and methoxy-chalcones are also contemplated. Chromium in specific form of polynicotinate or picolinate and other molecular forms that deliver bioactive chromium are also usefully included with the compositions of the present invention. Synergistic with this effect is the metal vanadium. The amounts of these supplemental and/or synergistic ingredients administered is an amount which effective to achieve the desired result based upon clinical experience after consideration of severity of the condition and physical assessment.

It is contemplated that in preferred aspects of the invention, the novel compositions contain specific mixtures of at least Hoodia and the GCBE. Other beneficial herbal ingredients of pharmaceutically acceptable ingredients will be additional and separate from the ratios provided herein for the two principal herbal ingredients. Generally, the ratio of Hoodia to green coffee bean extract is from about 99:1 to about 10:90 by weight. Preferably, it is from about 75:25 to about 25:75 by weight and, in some alternative aspects it is about 50:50 by weight.

The compositions of the present invention are administered as part of a pharmaceutically acceptable dosage form and/or orally acceptable vehicle such as those well known to those of ordinary skill. These include, without limitation, compressed tablets, capsules, powdered mixtures, granulations, gummy-type confectionery such as bears or other suitable shapes, chews, orally-acceptable liquid dosage forms including powered dietary shakes for reconstitution with a liquid or ready to drink shakes available from for example Slimfast®, Atkins®, Weight watcher®, etc., effervescent tablets, fast-dissolving orally dissolvable wafers or films such as those described in U.S. Pat. No. 6,596,298, the disclosure of which is incorporated herein by reference, oral sprays, etc. as will be apparent to those of ordinary skill. Regardless of the dosage form, the amount of Hoodia present is generally from about 5 to about 1,000 milligrams, preferably from about 100 to about 700 milligrams and more preferably from about 150 to about 250 milligrams per dose. The GCBE is present generally in amounts from about 10 to about 1000 milligrams, preferably from about 100 to about 500 milligrams, and more preferably from about 100 to about 150 milligrams per dose, depending upon the content of chlorogenic acid therein, it being understood that such extracts are commonly available containing either 10% or 50% by weight chlorogenic acid.

In alternative aspects of the invention, the compositions of the present invention are included as part of controlled release tablets, designed for once daily administration. Such formulations are made using well known techniques using controlled release polymer-based coatings such as those available from Colorcon under the Surelease® or Acryl-Eze® trademarks or those of other well-known vendors. Still further formulations, such as those designed for administration as a bulk powder for admixture to a liquid such as water, include soluble fiber obtained from vegetable sources such as oat bran (oat beta glucan) psyllium or microcrystalline cellulose.

Still further aspects of the invention provide compositions containing Hoodia and GCBE in the amounts shown above in further combination with one or more auxiliary therapeutic herbal or pharmaceutically derived active ingredients. A non-limiting list of suitable ingredients that are included for their known beneficial effects include those having stimulatory or metabolism increasing properties, blood pressure and/or cholesterol reducing effects, the ability to counteract insulin resistance, etc. For example, the compositions can include green tea polyphenols and other antioxidants, micronutrients, selected mineral and elements such as vitamins, chromium, vanadium, etc. fish oil, omega 3 fatty acids, alpha lipoic acid, Satietrol and other cholecystokinin (CCK) stimulators, milk thistle, as well as a wide variety of plant species and extracts thereof that may have a favorable impact on hyperglycemia or otherwise regulate blood sugar by supporting the body in improving glucose metabolism and activating glucose transport and uptake. Examples include but are not limited to Lagerstroemia speciosa L. and related botanicals and extracts, hydroxycitric acid, bitter melon, Ayurevedic herbs, linoleic acid, mixtures thereof, etc.

Further supplemental ingredients which can be included in the compositions of the present invention include 1-carnitene, kitosin, starch blockers such as white kidney bean (phaseolus vulgaris), sources of viscous soluble fiber including oat bran derived oat beta glucan, etc.

Another important aspect of the invention is a method of inducing weight loss in mammals, particularly humans. The method includes administering an effective amount of a Hoodia-GCBE composition described herein to a mammal in need of such treatment. It is contemplated that the compositions of the present invention will be administered at least once a day. Alternatively, the compositions can be administered as part of a weight controlling or other treatment regimen and be administered either before meals or as a meal replacement such as a milk-shake or other smoothie type beverage. It is also contemplated that the compositions will be included as part of snack bars or other processed foods as part of an overall weight control plan.

The present invention also includes a method of controlling appetite in mammals by administering an appetite suppressing amount of a compositions described herein to a mammal in need of such treatment. The amount given will depend on the expected variables known to the artisan including patient-related physical characteristics, etc. severity of condition and the like. In most aspects, doses of the compositions such as those described in the examples can be administered 1-4 times per day.

In another aspect of the invention, there are provided compositions containing pharmacologic-based ingredients. One preferred embodiment includes a) a Hoodia gordonii extract; and b) chlorogenic acid. The extracts contemplated for use herein can be those identified in the aforementioned CSIR's U.S. Pat. No. 6,376,657. More preferably, they are sterol glycosides such that as P57 or the compound of the formula:

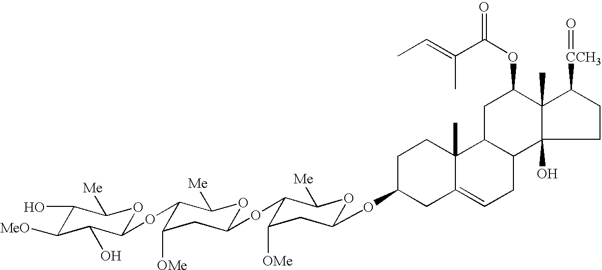

or derivatives or analogues thereof.

The compositions may further comprise an anti-hyperglycemic compound which include, without limitation, oral sulfonylureas and related compounds such as glypizide, glyburide, rosiglitazone, metformin, insulin and combinations thereof.

From the foregoing, it follows as a related aspect of this embodiment to provide a method of treating Type II diabetes mellitus. The method includes administering an effective amount of a composition described herein, namely the herbal-based Hoodia gordonii-GCBE combination or a combination of the specific extracts P57 and chlorogenic acid to a patient in need thereof. As was the case with the other methods of treatment described herein the amount of the compositions described herein will depend upon several factors, including the severity of the disease, size, weight, age, etc. of the patient. It is contemplated that the compositions of the invention will be administered from 1 to about 4 times a day.

Still further aspects of the invention include methods of increasing the effectiveness of an anti-diabetic compound in mammals. The methods include comprising administering an effective amount of a composition described herein in combination with the anti-diabetic compound to a mammal in need of such treatment. It will be understood that the phrase “in combination with” means not only as part of the same pharmaceutical dosage form but also separate dosage forms given in combination and not necessarily concomitantly (i.e. at different parts of the day) as part of a recognized therapeutic treatment regimen. Kits containing the combinations optionally with instructions for use in the treatment of mammals are also contemplated.

In view of the forgoing, a non-limiting list of suitable anti-diabetic compounds include glypizide, glyburide, rosiglitazone, metformin, insulin, as well as those pharmacologic agents know to those of ordinary skill, and combinations thereof.

The novel Hoodia-containing combinations described herein have a complementary effect on many medicines used in the treatment of diabetes mellitus (type II), the metabolic syndrome X, obesity and related conditions. See also Holt, Stephen, Supreme Properties of Hoodia Gordonii, Wellness Publishing, Inc. www.wellnesspublishing.com, (2005) Little Falls, N.J., the contents of which are incorporated herein by reference.

In a still further aspect of the invention there are provided additional compositions and methods of treatment which include not only the Hoodia-containing compositions described above but also an effective amount of one or more Caralluma extracts.

The Caralluma group of plants belongs to the Asclepiadaceae family and comprises a number of species that are distributed throughout the world. Some of these species include but are not limited to: c. indica, c. fimbriata, particularly preferred) c. attenuata, c. tuberculata, c. edulis, c. adscendens, c. stalagmifera, c. umbellata, c. penicillata, c. russeliana, c. retrospiciens, c. arabica and c. lasiantha. Some of the species are distributed throughout various parts of India.

Caralluma plants are small, erect and fleshy. They have 4 grooved stems that are almost round in shape. They are generally devoid of leaves and form small flowers in a variety of dark colors. Their pods are erect, linear and about 2.5 cms in length and feel velvety to the touch. The thorns of Caralluma are soft. The species of Caralluma found in India are edible and form part of the traditional medicine system of the country.

The medicinal properties of Caralluma have been attributed to the glycosides contained therein. A glycoside is a condensation product obtained from a sugar and non-sugar compound and may have further components such as ring structures that are substituted or non-substituted. The glycosides contained in Caralluma belong to the pregnane group of glycosides. Some of the pregnane group of glycosides found in Caralluma plants include, but are not limited to: caratuberside A, caratuberside B, bouceroside I, bouceroside II, bouceroside III, bouceroside IV, bouceroside V, bouceroside VI, bouceroside VII, bouceroside VIII, bouceroside IX, and bouceroside X. Such Caralluma extracts are available from commercial sources such as Genco Pacific of Texas and using the techniques described in US Patent Application Publication Number 2005/0202103A1, the contents of which are incorporated herein by reference. Alternative techniques of providing the extracts will be apparent to those of ordinary skill without undue experimentation.

In preferred aspects of this embodiment, the Caralluma extract includes at 5% w/w and preferably up to about 15% w/w or more of pregnane glycosides. One particularly preferred aspect of this embodiment includes effective amounts of Hoodia gordonii, green coffee bean extract containing chlorogenic acid and a mixture of Caralluma extracts which contains the pregnane glycosides caratuberside and bouceroside. The amount of the Caralluma extract included in the compositions of the present invention will vary somewhat depending upon several factors which will be apparent to the skilled artisan, including but limited to, the condition or syndrome being treated, the specific Caralluma extract(s) being included, the amount of Hoodia and GCBE being administered, etc. Without wishing to be limited to any specific dosage, it is believed that amounts of from about 10 mg to about 1 gram per dose given from one to up several (i.e. six or more) times a day. Compositions including the Hoodia, GCBE and Caralluma can have a ratio of Hoodia to green coffee bean extract to Caralluma extract which ranges from about 90:5:5 to about 33:33:33 by weight.

The specification makes frequent use of the term “mammals” in describing the methods of treatment. It will be understood that humans are the preferred mammals being treated.

### EXAMPLES

The following examples serve to provide further appreciation of the invention but are not meant in any way to restrict the effective scope of the invention.

### Example 1

Base Capsule Formulation

| | | Ranges Mg |
|---|---|---|
| Ingredient | Mg per dosage | (min) to (max) |
| Hoodia gordonii (obtained as | 200 | 100-700 |
| powdered from whole plant) | | |
| Green coffee bean extract | 120 | 100-500 |
| Pharmaceutical Excipients | QS | |

The ingredients are combined using pharmaceutically acceptable techniques to achieve a uniform blend of the ingredients and encapsulated using standard state of the art encapsulation equipment.

It is contemplated that such capsules are or can be administered to patients in need thereof 1-4 times per day.

### Example 2

Enhanced Capsule Formulations

| Ingredient | Mg per dosage (range) |
|---|---|
| Hoodia gordonii (obtained as | 100-700 |
| powdered from whole plant) | |
| Green coffee bean extract | 100-500 |
| Green tea polyphenols | 10-100 mg |
| Soluble fiber | 50-500 mg |
| chromium | USDA requirements |
| minerals and micronutrients | 1-500 mg in total |
| Pharmaceutical Excipients | QS |

The capsules are prepared in the same way as described in Example 1.

### Examples 3-4

### Powdered Formulations for Milk Shake

The formulations of examples 1 and 2 are left in powdered form and admixed to a standard dietary shake formulation such as Slim Fast or a related product so that a scoop-measure thereof provides the amounts of the Hoodia and GCBE mentioned herein.

### Examples 5-6

The procedures of Examples 1-2 are repeated except that 200-400 mg of Caralluma fimbriata extract obtained from Gencor Pacific is included in each capsule type.

### Example 7

### Hoodia Supreme Plus Tablets

In this example, compressed tablets were made to according to the following formula:

| | Mg per dosage |
|---|---|
| Ingredient | (range) +/−5% |
| Hoodia gordonii (aerial parts) | 200 |
| Green coffee bean extract (52-58% | 75 |
| chlorogenic acid) | |
| Green tea leaf extract providing | 400 mg |
| ˜90 mg ECGC | |
| Pharmaceutical Excipients: including | QS |
| Dicalcium Phosphate, | |
| Microcrystalline Cellulose, | |
| Croscarmellose Sodium, Stearic | |
| Acid, Magnesium Stearate, Silica, | |
| Pharmaceutical Glaze | |

### Example 8

In this example, a protocol for treating metabolic Syndrome X with a composition of the present invention is described. Tablets of Example 7, available from Natures Benefit, Little Falls, N.J., as Hoodia Supreme Plus are obtained. Patients suffering from metabolic Syndrome X are given physical exams, weighed, baseline blood pressure, blood cholesterol and fasting blood glucose (hereinafter “parameters”) are measured. Each patient is administered 1 tablet with water 3 times a day for 1 month. Although no other formal restrictions are placed on the members of the group, each member is encouraged to make healthy improvements in their diet, exercise, lifestyle habits. At the end of a month trial period, physical exams and all lab work is repeated to observe the decrease in each of the parameters. Depending upon the reduction observed, the dosage of the tablets is adjusted to a maintenance regimen or more aggressive therapy.

### Example 9

In this example six volunteer obese patients, 2 male and 4 female undergo an assessment of the efficacy of the tablets of Example 7 in the short term management of their obesity. Their body-mass indices range from 25-33. Each patient takes two tablets daily with an 8 ounce glass of water daily for one month. Each patient is asked to describe their appetite and desire for food. Each indicates a decrease in appetite and desire for food. Compliance is monitored and the therapy results in average weight loss of about 3 pounds per week.

Other embodiments of the invention will be apparent to one skilled in the art from a consideration of this specification or practice of the invention disclosed herein. It is intended that the specification and examples be considered as exemplary only, with the true scope and spirit of the invention being indicated by the following claims.

The inventor has open label research experience of improvements in disease states and metabolic parameters in individuals with obesity, the metabolic syndrome, diabetes mellitus and related disorders.

What is claimed is:

1. A composition comprising
a) a weight reducing amount of Hoodia gordonii or an extract thereof; and
b) an effective amount of green coffee bean extract comprising chlorogenic acid.

2. The composition of claim 1, wherein the ratio of Hoodia to green coffee bean extract is from about 99:1 to about 10:90 by weight.

3. The composition of claim 2, wherein the ratio of Hoodia to green coffee bean extract is from about 75:25 to about 25:75 by weight.

4. The composition of claim 3, wherein the ratio of Hoodia to green coffee bean extract is from about 50:50 by weight.

5. The composition of claim 1, wherein the Hoodia gordonii is in powdered form derived from the whole Hoodia gordonii plant.

6. The composition of claim 1, further comprising an effective amount a Caralluma extract.

7. The composition of claim 6, wherein the ratio of Hoodia to green coffee bean extract to Caralluma extract ranges from about 90:5:5 to about 33:33:33 by weight.

8. The composition of claim 6, further comprising a member of the groups consisting of green tea leaf extract, chromium picolate, cinnamon, methyl hydroxy chalcone polymer (MHCP), vanadium and mixtures thereof.

9. A pharmaceutically acceptable dosage form comprising a composition of claim 1 and a sufficient amount of a pharmaceutically acceptable excipient.

10. The pharmaceutically acceptable dosage form of claim 9, wherein the Hoodia is present in an amount of from about 5 to about 200 milligrams.

11. The pharmaceutically acceptable dosage form of claim 10, wherein the Hoodia is present in an amount of from about 5 to about 100 milligrams.

12. The pharmaceutically acceptable dosage form of claim 11, wherein the Hoodia is present in an amount of from about 10 to about 80 milligrams.

13. The pharmaceutically acceptable dosage form of claim 9, wherein the green coffee bean extract is present in an amount of from about 10 to about 1000 milligrams.

14. The pharmaceutically acceptable dosage form of claim 13, wherein the green coffee bean extract is present in an amount of from about 100 to about 750 milligrams.

15. The pharmaceutically acceptable dosage form of claim 14, wherein the green coffee bean extract is present in an amount of from about 200 to about 600 milligrams.

16. The pharmaceutically acceptable dosage form of claim 9, further comprising from about 10 mg to about 1,000 milligrams of a Caralluma extract.

17. A pharmaceutically acceptable dosage form of claim 9, selected from the group consisting of compressed tablets, capsules, powdered mixtures, orally-acceptable liquid dosage forms, confectionery forms, dissolvable oral strips and effervescent tablets.

18. A method of inducing weight loss or treating metabolic Syndrome X in mammals, comprising administering an effective amount of a composition of claim 1 to a mammal in need of such treatment.

19. A method of controlling appetite in mammals, comprising administering an appetite suppressing amount of a composition of claim 1 to a mammal in need thereof.

20. A composition comprising a) a weight reducing amount of Hoodia gordonii or an extract thereof; and b) an effective amount of chlorogenic acid.

21. The composition of claim 22, wherein the extract of Hoodia gordonii is a sterol glycoside.

22. The composition of claim 20, further comprising an anti-hyperglycemic compound.

23. The composition of claim 22, wherein the anti-hyperglycemic compound is selected from the group consisting of glypizide, glyburide, rosiglitazone, metformin, insulin and combinations thereof.

24. A method of treating Type II diabetes mellitus, comprising administering an effective amount of a composition of claim 1 to a patient in need thereof.

25. A method of increasing the effectiveness of an anti-diabetic compound in mammals, comprising administering an effective amount of a composition of claims 1 in combination with said anti-diabetic compound to a mammal in need of such treatment.

26. The method of claim 25, wherein the anti-diabetic compound is selected from the group consisting of oral sulfonylureas and anti-hyperglycemics.
